(19) United States
(12) Patent Application Publication
(10) Pub. No.: US 2017/0356025 A1
(43) Pub. Date: Dec. 14, 2017

(54) INTERNAL CONTROL PROBES FOR IMPROVING PCR ASSAY PERFORMANCE

(71) Applicant: Roche Molecular Systems, Inc., Pleasanton, CA (US)

(72) Inventor: Kalyani Mangipudi, Pleasanton, CA (US)

(21) Appl. No.: 15/618,450

(22) Filed: Jun. 9, 2017

Related U.S. Application Data
(60) Provisional application No. 62/349,810, filed on Jun. 14, 2016.

(51) Int. Cl. C12Q 1/68; C12Q 1/70

(52) U.S. Cl. CPC C12Q 1/686; C12Q 1/6893; C12Q 1/689; C12Q 1/6825; C12Q 1/701; C12Q 2600/166; C12Q 2600/16

(57) ABSTRACT

The present invention relates to methods for improving multiplex real-time PCR assays by the use of an internal control probe labeled with Quasar 705.

### CROSS REFERENCE TO RELATED APPLICATIONS

This application claims the benefit of priority of U.S. Provisional Patent Application Ser. No. 62/349,810, filed Jun. 14, 2016, which is incorporated herein by reference in its entirety.

### FIELD OF THE INVENTION

The present invention relates to the field of molecular diagnostics. Within this field, the invention particularly concerns PCR amplification of one or more target nucleic acids that may be present in a sample in the presence of an internal control nucleic acid and internal control probes that are used to detect the internal control nucleic acid.

### BACKGROUND OF THE INVENTION

In the field of molecular diagnostics, the amplification of nucleic acids from numerous sources has been of considerable significance. Examples for diagnostic applications of nucleic acid amplification and detection are the detection of viruses such as Human Papilloma Virus (HPV), West Nile Virus (WNV) or the routine screening of blood donations for the presence of Human Immunodeficiency Virus (HIV), Hepatitis-B (HBV) and/or C Virus (HCV). Furthermore, said amplification techniques are suitable for bacterial targets such as Mycoplasma genitalium (MG), protozoan targets such as Trichomonas vaginalis (TV) or the analysis of oncology markers.

The most prominent and widely-used amplification technique is Polymerase Chain Reaction (PCR). Other amplification reactions comprise, among others, the Ligase Chain Reaction, Polymerase Ligase Chain Reaction, Gap-LCR, Repair Chain Reaction, 3SR, NASBA, Strand Displacement Amplification (SDA), Transcription Mediated Amplification (TMA), and QP-amplification.

Automated systems for PCR-based analysis often make use of real-time detection of product amplification during the PCR process in the same reaction vessel. Key to such methods is the use of modified oligonucleotides carrying reporter groups or labels.

It has been shown that amplification and detection of more than one target nucleic acid in the same vessel is possible. This method is commonly termed “multiplex” amplification and requires different labels for distinction if real-time detection is performed. In a multiplex real-time PCR assay, each probe oligonucleotide that is specific for a given target nucleic acid is labeled with a specific signal generating moiety (e.g. a fluorescent dye) and the detection of the presence or absence of a given signal reflects the presence or absence of the given target nucleic acid within the reaction vessel.

It is mostly desirable or even mandatory in the field of clinical nucleic acid diagnostics to control the respective amplification using control nucleic acids with a known sequence, for qualitative (performance control) and/or quantitative (determination of the quantity of a target nucleic using the control as a reference) purposes. Given the diversity especially of diagnostic targets, comprising prokaryotic, eukaryotic as well as viral nucleic acids, and given the diversity between different types of nucleic acids such as RNA and DNA, control nucleic acids are usually designed in a specific manner. This circumstance applies for both qualitative and quantitative assays.

In general one can distinguish external and internal controls. External controls, like classical positive and negative controls, mimic positive and negative samples and are normally used to check whether the assay runs properly or whether contaminants are contained. An internal control for example is useful for recognizing inhibitory substances possibly contained in a sample or can be used as a quantification standard in a quantitative assay. In contrast to an external control, which normally is tested in a separate reaction chamber, an internal control is preferably incubated in the same reaction vessel together with the target nucleic acid to be tested. Therefore, the control or the amplified product of that control has to be distinguishable from the target nucleic acid or from the amplified product of that target nucleic acid. When using an amplification method an internal control nucleic acid is being co-amplified essentially under the same reaction conditions as the target nucleic acid. These conditions include reagent concentrations, temperature, inhibitor concentration or enzymatic activities. Frequently used sequences for controls are derived from housekeeping genes (see Chelly, J., et al., Eur. J. Biochem. 187 (1990) 691-698; Mallet, F., et al., J. Clin. Microbiol. 33 (1995) 3201-3208), but also non-natural sequences are being used (see e.g. EP 1 236 805).

Because the internal control nucleic acid is present in the reaction vessel comprising the target nucleic acid and is expected to provide a signal, both the internal control nucleic acid and the signal thereof has to be differentiable from the target nucleic acid(s) and the signal(s) thereof. Ideally, the signal from the internal control should not interfere with the signal(s) from the target nucleic acid(s).

### BRIEF SUMMARY OF THE INVENTION

The present invention relates to the surprising observation that the use of a Quasar 705-labeled internal control probe in a multiplex real-time PCR assay leads to overall improvement in assay performance. Specifically, the sensitivity in the signal from the FAM and HEX channels were boosted. A remarkable increase in relative fluorescence intensity (RFI) was observed in the HEX channel when a Quasar 705-labeled internal control probe was used in the multiplex as compared to when a Cy5.5-labeled internal control probe was used. In the FAM channel, the Quasar 705-labeled internal control probes's presence increased the sensitivity and allowed lower levels of detection previously not possible with the Cy5.5-labeled probe in the multiplex assay. Furthermore, the signals generated from Quasar 705 were more robust and tighter than signals from Cy5.5.

Therefore in one aspect, the present invention provides for a method for detecting two or more target nucleic acids in a sample comprising the steps of: a) providing a reaction mixture comprising an internal control (IC) nucleic acid; two or more target-specific primer pairs that hybridize to distinct sequence portions of the two or more target nucleic acid; an IC-specific primer pair that hybridize to distinct sequence portions of the IC nucleic acid; two or more target-specific probes wherein each one target-specific probe is labeled with a fluorescent dye that is different from another target-specific probe, and wherein each one of the two or more target-specific probes specifically hybridize to each one of the two or more target nucleic acid sequences amplified by each one of the two or more target-specific primer pairs; an IC-specific probe labeled with Quasar 705 that hybridizes to the IC nucleic acid sequence amplified by the IC-specific primer pair; b) adding the sample to the reaction mixture; c) performing one or more cycling steps, wherein each cycling step comprises: an amplifying step comprising producing two or more amplification products derived from the two or more target nucleic acids if present in the sample and producing an amplification product derived from the IC nucleic acid, and a hybridizing step comprising hybridizing amplification products with probes to generate fluorescent signals; d) detecting and measuring signals generated from each fluorescent dye on the two or more target-specific probes and from Quasar 705 on the IC-specific probe in step c), wherein the presence or absence of fluorescent signals generated from the target-specific probes are indicative of the presence or absence of the target nucleic acids; wherein the sensitivity and intensity of the signals generated from the fluorescent dyes on the target-specific probes is improved when the IC-specific probe is labeled with Quasar 705 than when the IC-specific probe is labeled with Cy5.5. In one embodiment, the fluorescent dye on each of the target-specific probes is a fluorescein dye, a rhodamine dye, a cyanine dye, and a coumarin dye. In some embodiments, the fluorescent dye on the target-specific probes is selected from Fluorescein (FAM), Hexachloro-fluorescein (HEX), JA270, CAL635, Coumarin343, Cyan500, CY5.5, LC-Red 640, and/or LC-Red 705. In certain embodiments, the fluorescent dye on the target-specific probes is selected from FAM and/or HEX. In some embodiments, any one or more of the primers and/or probes comprises a modified nucleotide or a non-nucleotide compound. In some embodiments, the two or more target nucleic acid sequences are from one or more DNA viruses or from one or more bacteria. In certain embodiments, the two or more target nucleic acid sequences are from Chlamydia trachomatis (CT), Neisseria gonorrhoeae (NG), Trichomonas vaginalis (TV) and/or Mycoplasma genitalium (MG). In certain embodiments, the two or more target nucleic acid sequences are from Trichomonas vaginalis (TV) and Mycoplasma genitalium (MG). In some embodiments, said internal control nucleic acid is DNA. In other embodiments, the two or more target nucleic acid sequences are from one or more RNA viruses. In certain embodiments, the one or more RNA viruses are selected from any one of Human Immunodeficiency Virus (HIV), Hepatitis C Virus (HCV), the West Nile Virus (WNV), Human Papilloma Virus (HPV), Japanese Encephalitis Virus (JEV), and/or St. Louis Encephalitis Virus (SLEV). In some embodiments, said internal control nucleic acid is RNA.

### BRIEF DESCRIPTION OF THE DRAWINGS

FIG. 1 shows the chemical structure of Quasar 705 (* represents attachment site to the nucleotide).

FIG. 2A shows the PCR growth curves generated by different starting concentrations of Trichomonas vaginalis (TV) genomic DNA measured as FAM signals in the presence of a Cy5.5-labeled internal control probe.

FIG. 2B shows the PCR growth curves generated by different starting concentrations of Trichomonas vaginalis (TV) genomic DNA measured as FAM signals in the presence of a Quasar 705-labeled internal control probe.

FIG. 3A shows the PCR growth curves generated by the internal control DNA using a Cy5.5-labeled internal control probe in the TV assay.

FIG. 3B shows the PCR growth curves generated by the internal control DNA using a Quasar 705-labeled internal control probe in the TV assay.

FIG. 4A shows the PCR growth curves generated by different starting concentrations of Mycoplasma genitalium (MG) genomic DNA measured as HEX signals in the presence of a Cy5.5-labeled internal control probe.

FIG. 4B shows the PCR growth curves generated by different starting concentrations of Mycoplasma genitalium (MG) genomic DNA measured as HEX signals in the presence of a Quasar 705-labeled internal control probe.

FIG. 5A shows the PCR growth curves generated by the internal control DNA using a Cy5.5-labeled internal control probe in the MG assay.

FIG. 5B shows the PCR growth curves generated by the internal control DNA using a Quasar 705-labeled internal control probe in the MG assay.

### DETAILED DESCRIPTION OF THE INVENTION

### I. Definitions

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The terms “a,” “an,” and “the” include plural referents, unless the context clearly indicates otherwise.

The disclosed methods may include performing at least one cycling step that includes amplifying one or more portions of a given nucleic acid molecule gene target from a sample using one or more pairs of primers. “Primer(s)” as used herein refer to oligonucleotide primers that specifically anneal to the target gene, and initiate DNA synthesis therefrom under appropriate conditions producing the respective amplification products. Each of the discussed primers anneals to a target within or adjacent to the respective target nucleic acid molecule such that at least a portion of each amplification product contains nucleic acid sequence corresponding to the target. The one or more amplification products are produced provided that one or more of the target gene nucleic acid is present in the sample, thus the presence of the one or more of target gene amplification products is indicative of the presence of the target nucleic acid in the sample. The amplification product should contain the nucleic acid sequences that are complementary to one or more detectable probes for target gene. “Probe(s)” as used herein refer to oligonucleotide probes that specifically anneal to nucleic acid sequence encoding the target gene. Each cycling step includes an amplification step, a hybridization step, and a detection step, in which the sample is contacted with the one or more detectable probes for detection of the presence or absence of the target nucleic acid in the sample.

As used herein, the term “amplifying” refers to the process of synthesizing nucleic acid molecules that are complementary to one or both strands of a template nucleic acid molecule. Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

“Amplification reagents”, in the context of the invention, are chemical or biochemical components that enable the amplification of nucleic acids. Such reagents comprise, but are not limited to, nucleic acid polymerases, buffers, mononucleotides such as nucleoside triphosphates, oligonucleotides e.g. as oligonucleotide primers, salts and their respective solutions, detection probes, dyes, and more.

The term “primer” as used herein is known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′-OH group whereto further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, or a “probe”.

The term “hybridizing” refers to the annealing of one or more probes to an amplification product. Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T ruber, T thermophilus, T aquaticus, T lacteus, T rubens, Bacillus stearothermophilus, and Methanothermus fervidus. Nonetheless, polymerases that are not thermostable also can be employed in PCR assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-0-methyl Ribo-U, 2′-0-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference.

A “modified oligonucleotide” (or “oligonucleotide analog”), belonging to another specific subgroup of oligomeric compounds, possesses one or more nucleotides and one or more modified nucleotides as monomeric units. Thus, the term “modified oligonucleotide” (or “oligonucleotide analog”) refers to structures that function in a manner substantially similar to oligonucleotides and can be used interchangeably in the context of the present invention. From a synthetical point of view, a modified oligonucleotide (or an oligonucleotide analog) can for example be made by chemical modification of oligonucleotides by appropriate modification of the phosphate backbone, ribose unit or the nucleotide bases (Uhlmann and Peyman, Chemical Reviews 90 (1990) 543; Verma S., and Eckstein F., Annu. Rev. Biochem. 67 (1998) 99-134). Representative modifications include phosphorothioate, phosphorodithioate, methyl phosphonate, phosphotriester or phosphoramidate inter-nucleoside linkages in place of phosphodiester internucleoside linkages; deaza- or azapurines and -pyrimidines in place of natural purine and pyrimidine bases, pyrimidine bases having substituent groups at the 5 or 6 position; purine bases having altered substituent groups at the 2, 6 or 8 positions or 7 position as 7-deazapurines; bases carrying alkyl-, alkenyl-, alkinyl or aryl-moieties, e.g. lower alkyl groups such as methyl, ethyl, propyl, butyl, tert-butyl, pentyl, hexyl, heptyl, octyl, nonyl, decyl, or aryl groups like phenyl, benzyl, naphtyl; sugars having substituent groups at, for example, their 2′ position; or carbocyclic or acyclic sugar analogs. Other modifications consistent with the spirit of this invention are known to those skilled in the art. Such modified oligonucleotides (or oligonucleotide analogs) are best described as being functionally interchangeable with, yet structurally different from, natural oligonucleotides. In more detail, exemplary modifications are disclosed in Verma S., and Eckstein F., Annu. Rev. Biochem. 67 (1998) 99-134 or WO 02/12263. In addition, modification can be made wherein nucleoside units are joined via groups that substitute for the internucleoside phosphate or sugar phosphate linkages. Such linkages include those disclosed in Verma S., and Eckstein F., Annu. Rev. Biochem. 67 (1998) 99-134. When other than phosphate linkages are utilized to link the nucleoside units, such structures have also been described as “oligonucleosides”.

A “nucleic acid” as well as the “target nucleic acid” is a polymeric compound of nucleotides as known to the expert skilled in the art. “Target nucleic acid” is used herein to denote a nucleic acid in a sample which should be analyzed, i.e. the presence, non-presence and/or amount thereof in a sample should be determined.

“Labels”, often referred to as “reporter groups”, are generally groups that make a nucleic acid, in particular oligonucleotides or modified oligonucleotides, as well as any nucleic acids bound thereto distinguishable from the remainder of the sample (nucleic acids having attached a label can also be termed labeled nucleic acid binding compounds, labeled probes or just probes). Exemplary labels according to the invention are fluorescent labels, which are e.g. fluorescent dyes such as a fluorescein dye, a rhodamine dye, a cyanine dye, and a coumarin dye. Exemplary fluorescent dyes according to the invention are Fluorescein (FAM), Hexachloro-fluorescein (HEX), JA270, CAL635, Coumarin343, Quasar705, Cyan500, CY5.5, LC-Red 640, LC-Red 705.
In the context of the invention, any primer and/or probe may be chemically modified, i.e. the primer and/or the probe comprise a modified nucleotide or a non-nucleotide compound. The probe or the primer is then a modified oligonucleotide.

A method of nucleic acid amplification is the Polymerase Chain Reaction (PCR) which is disclosed, among other references, in U.S. Pat. Nos. 4,683,202, 4,683,195, 4,800,159, and 4,965,188. PCR typically employs two or more oligonucleotide primers that bind to a selected nucleic acid template (e.g. DNA or RNA). Primers useful for nucleic acid analysis include oligonucleotides capable of acting as a point of initiation of nucleic acid synthesis within the nucleic acid sequences of the target nucleic acids. A primer can be purified from a restriction digest by conventional methods, or it can be produced synthetically. The primer can be single-stranded for maximum efficiency in amplification, but the primer can be double-stranded. Double-stranded primers are first denatured, i.e., treated to separate the strands. One method of denaturing double stranded nucleic acids is by heating. A “thermostable polymerase” is a polymerase enzyme that is heat stable, i.e., it is an enzyme that catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have e.g. been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 5 sec to 9 min. In order to not expose the respective polymerase like e.g. the Z05 DNA Polymerase to such high temperatures for too long and thus risking a loss of functional enzyme, it can be preferred to use short denaturation steps.

If the double-stranded template nucleic acid is denatured by heat, the reaction mixture is allowed to cool to a temperature that promotes annealing of each primer to its target sequence on the target nucleic acids.

The temperature for annealing can be from about 35° C. to about 70° C., or about 45° C. to about 65° C.; or about 50° C. to about 60° C., or about 55° C. to about 58° C. Annealing times can be from about 10 sec to about 1 min (e.g., about 20 sec to about 50 sec; about 30 sec to about 40 sec). In this context, it can be advantageous to use different annealing temperatures in order to increase the inclusivity of the respective assay. In brief, this means that at relatively low annealing temperatures, primers may also bind to targets having single mismatches, so variants of certain sequences can also be amplified. This can be desirable if e.g. a certain organism has known or unknown genetic variants which should also be detected. On the other hand, relatively high annealing temperatures bear the advantage of providing higher specificity, since towards higher temperatures the probability of primer binding to not exactly matching target sequences continuously decreases. In order to benefit from both phenomena, in some embodiments of the invention the process described above comprises annealing at different temperatures, for example first at a lower, then at a higher temperature. If, e.g., a first incubation takes place at 55° C. for about 5 cycles, non-exactly matching target sequences may be (pre-)amplified. This can be followed e.g. by about 45 cycles at 58° C., providing for higher specificity throughout the major part of the experiment. This way, potentially important genetic variants are not missed, while the specificity remains relatively high.

The reaction mixture is then adjusted to a temperature at which the activity of the polymerase is promoted or optimized, i.e., a temperature sufficient for extension to occur from the annealed primer to generate products complementary to the nucleic acid to be analyzed. The temperature should be sufficient to synthesize an extension product from each primer that is annealed to a nucleic acid template, but should not be so high as to denature an extension product from its complementary template (e.g., the temperature for extension generally ranges from about 40° to 80° C. (e.g., about 50° C. to about 70° C.; about 60° C.). Extension times can be from about 10 sec to about 5 min, or about 15 sec to 2 min, or about 20 sec to about 1 min, or about 25 sec to about 35 sec. The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acids. The limiting factors in the reaction are the amounts of primers, thermostable enzyme, and nucleoside triphosphates present in the reaction. The cycling steps (i.e., denaturation, annealing, and extension) can be repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

Within the scope of the invention, a PCR can be carried out in which the steps of annealing and extension are performed in the same step (one-step PCR) or, as described above, in separate steps (two-step PCR). Performing annealing and extension together and thus under the same physical and chemical conditions, with a suitable enzyme such as, for example, the Z05 DNA polymerase, bears the advantage of saving the time for an additional step in each cycle, and also abolishing the need for an additional temperature adjustment between annealing and extension. Thus, the one-step PCR reduces the overall complexity of the respective assay.

The internal control nucleic acid used in the present invention may exhibit the following properties relating to its sequence:

a melting temperature from 55° C. to 90° C., or from 65° C. to 85° C., or from 70° C. to 80° C., or about 75° C.

a length of up to 500 bases or base pairs, or from 50 to 300 bases or base pairs, or from 100 to 200 bases or base pairs, or about 180 bases or base pairs

a GC content from 30% to 70%, or from 40% to 60%, or about 50%.

In the context of the invention, a “sequence” is the primary structure of a nucleic acid, i.e. the specific arrangement of the single nucleobases of which the respective nucleic acids consists. It has to be understood that the term “sequence” does not denote a specific type of nucleic acid such as RNA or DNA, but applies to both as well as to other types of nucleic acids such as e.g. PNA or others. Where nucleobases correspond to each other, particularly in the case of uracil (present in RNA) and thymine (present in DNA), these bases can be considered equivalent between RNA and DNA sequences, as well-known in the pertinent art.

Clinically relevant nucleic acids are often DNA which can be derived e.g. from DNA viruses like e.g. Hepatitis B Virus (HBV), Cytomegalovirus (CMV) and others, or bacteria like e.g. Chlamydia trachomatis (CT), Neisseria gonorrhoeae (NG) and others. In such cases, it can be advantageous to use an internal control nucleic acid consisting of DNA, in order to reflect the target nucleic acids properties. Therefore, an aspect of the invention is the method described above, wherein said internal control nucleic acid is DNA.

On the other hand, numerous nucleic acids relevant for clinical diagnostics are ribonucleic acids, like e.g. the nucleic acids from RNA viruses such as for example Human Immunodeficiency Virus (HIV), Hepatitis C Virus (HCV), the West Nile Virus (WNV), Human Papilloma Virus (HPV), Japanese Encephalitis Virus (JEV), St. Louis Encephalitis Virus (SLEV) and others. The present invention can be readily applied to such nucleic acids. In this case, it can be advantageous to use an internal control nucleic acid consisting of RNA, in order to reflect the target nucleic acids properties. If both RNA and DNA are to be analyzed in the process described supra, the internal control nucleic acid can be RNA, as the internal control nucleic acid mimics the most sensitive target of an assay involving multiple targets, and RNA targets usually have to be more closely controlled.

Thus, an aspect of the invention is the method described above, wherein said internal control nucleic acid is RNA.

Since RNA is more prone to degradation than DNA due to influences such as alkaline pH, ribonucleases etc., internal control nucleic acids made of RNA may be provided as armored particles. Armored particles such as especially armored RNA are described e.g. in EP910643. In brief, the RNA, which can be produced chemically or heterologously e.g. by bacteria such as e.g. E. coli, is at least partially encapsulated in a viral coat protein. The latter confers resistance of the RNA towards external influences, in particular ribonucleases. It must be understood that internal control DNA can also be provided as an armored particle. Both armored RNA and DNA are useful as internal control nucleic acids in the context of the invention. In an embodiment, RNA control nucleic acids are armored with the MS2 coat protein in E. coli. In a further embodiment, DNA control nucleic acids are armored using lambda phage GT11. Therefore, an aspect of the invention is the method described above, wherein said internal control nucleic acid is an armored nucleic acid.

Typically, in amplification-based nucleic acid diagnostics, RNA templates are transcribed into DNA prior to amplification and detection. Hence, an aspect of the invention is the process described above, wherein said amplification reagents comprise a polymerase with reverse transcriptase activity, said process further comprising between step e. and step f the step of incubating in said reaction vessels said purified nucleic acids with said one or more amplification reagents for a period of time and under conditions suitable for transcription of RNA by said polymerase with reverse transcriptase activity to occur.

A “polymerase with reverse transcriptase activity” is a nucleic acid polymerase capable of synthesizing DNA based on an RNA template. It is also capable of the formation of a double-stranded DNA once the RNA has been reverse transcribed into a single strand cDNA. In an embodiment of the invention, the polymerase with reverse transcriptase activity is thermostable.

In an embodiment, the process according to the invention comprises incubating a sample containing an RNA template with an oligonucleotide primer sufficiently complementary to said RNA template to hybridize with the latter, and a thermostable DNA polymerase in the presence of at least all four natural or modified deoxyribonucleoside triphosphates, in an appropriate buffer comprising a metal ion buffer which, in an embodiment, buffers both the pH and the metal ion concentration. This incubation is performed at a temperature sufficient for said primer to hybridize to said RNA template and said DNA polymerase to catalyze the polymerization of said deoxyribonucleoside triphosphates to form a cDNA sequence complementary to the sequence of said RNA template.

As used herein, the term “cDNA” refers to a complementary DNA molecule synthesized using a ribonucleic acid strand (RNA) as a template. The RNA may e.g. be mRNA, tRNA, rRNA, or another form of RNA, such as viral RNA. The cDNA may be single-stranded, double-stranded or may be hydrogen-bonded to a complementary RNA molecule as in an RNA/cDNA hybrid.

A primer suitable for annealing to an RNA template may also be suitable for amplification by PCR. For PCR, a second primer, complementary to the reverse transcribed cDNA strand, provides an initiation site for the synthesis of an extension product.

In the amplification of an RNA molecule by a DNA polymerase, the first extension reaction is reverse transcription using an RNA template, and a DNA strand is produced. The second extension reaction, using the DNA template, produces a double-stranded DNA molecule. Thus, synthesis of a complementary DNA strand from an RNA template by a DNA polymerase provides the starting material for amplification.

Thermostable DNA polymerases can be used in a coupled, one-enzyme reverse transcription/amplification reaction. The term “homogeneous”, in this context, refers to a two-step single addition reaction for reverse transcription and amplification of an RNA target. By homogeneous it is meant that following the reverse transcription (RT) step, there is no need to open the reaction vessel or otherwise adjust reaction components prior to the amplification step. In a non-homogeneous RT/PCR reaction, following reverse transcription and prior to amplification one or more of the reaction components such as the amplification reagents are e.g. adjusted, added, or diluted, for which the reaction vessel has to be opened, or at least its contents have to be manipulated. Both homogeneous and non-homogeneous embodiments are comprised by the scope of the invention.

Reverse transcription is an important step in an RT/PCR. It is, for example, known in the art that RNA templates show a tendency towards the formation of secondary structures that may hamper primer binding and/or elongation of the cDNA strand by the respective reverse transcriptase. Thus, relatively high temperatures for an RT reaction are advantageous with respect to efficiency of the transcription. On the other hand, raising the incubation temperature also implies higher specificity, i.e. the RT primers will not anneal to sequences that exhibit mismatches to the expected sequence or sequences. Particularly in the case of multiple different target RNAs, it can be desirable to also transcribe and subsequently amplify and detect sequences with single mismatches, e.g. in the case of the possible presence of unknown or rare substrains or subspecies of organisms in the fluid sample.

As a further important aspect of reverse transcription, long RT steps can damage the DNA templates that may be present in the fluid sample. If the fluid sample contains both RNA and DNA species, it is thus favorable to keep the duration of the RT steps as short as possible, but at the same time ensuring the synthesis of sufficient amounts of cDNA for the subsequent amplification and optional detection of amplificates.

Particularly suitable for these requirements are enzymes carrying a mutation in the polymerase domain that enhances their reverse transcription efficiency in terms of a faster extension rate. Therefore, an aspect of the invention is the process described above, wherein the polymerase with reverse transcriptase activity is a polymerase comprising a mutation conferring an improved nucleic acid extension rate and/or an improved reverse transcriptase activity relative to the respective wildtype polymerase. In an embodiment, in the process described above, the polymerase with reverse transcriptase activity is a polymerase comprising a mutation conferring an improved reverse transcriptase activity relative to the respective wildtype polymerase.

Polymerases carrying point mutations that render them particularly useful in the context of the invention are disclosed in WO 2008/046612. One example is mutants of the thermostable DNA polymerase from Thermus species Z05 (described e.g. in U.S. Pat. No. 5,455,170), said variations comprising mutations in the polymerase domain as compared with the respective wildtype enzyme Z05. An embodiment for the method according to the invention is a mutant Z05 DNA polymerase wherein the amino acid at position 580 is selected from the group consisting of G, T, R, K and L.

For reverse transcription using a thermostable polymerase, Mn²⁺ can bes the divalent cation and is typically included as a salt, for example, manganese chloride (MnC¹₂), manganese acetate (Mn(OAc)₂), or manganese sulfate (MnSO₄). If MnCl₂ is included in a reaction containing 50 mM Tricine buffer, for example, the MnCl₂ is generally present at a concentration of 0.5-7.0 mM; 0.8-1.4 mM is preferred when 200 mM of each dGTP, dATP, dUTP, and, dCTP are utilized; and 2.5-3.5 mM MnCl₂ is most preferred. Further, the use of Mg²⁺ as a divalent cation for reverse transcription is also in the context of the present invention.

Since it is in the scope of the invention to reverse-transcribe RNA target nucleic acids into cDNA while preserving the DNA target nucleic acids so both cDNA and DNA can be used for subsequent amplification, the internally controlled process described above is particularly useful for the simultaneous amplification of target nucleic acids derived from both organisms having an RNA or organisms having a DNA genome. This advantage considerably increases the spectrum of different organisms, especially pathogens, that can be analyzed under identical physical conditions. Therefore, an aspect of the invention is the process described above, wherein the at least two target nucleic acids comprise RNA and DNA.

An “organism”, as used herein, means any living single- or multicellular life form. In the context of the invention, a virus is an organism.

Especially due to an appropriate temperature optimum, enzymes like Tth polymerase or, for example, the mutant ZO5 DNA polymerase mentioned above are suited to carry out the subsequent step of amplification of the target nucleic acids. Exploiting the same enzyme for both reverse transcription an amplification contributes to the ease of carrying out the process and facilitates its automation, since the fluid sample does not have to be manipulated between the RT and the amplification step.

The target of the amplification step can be an RNA/DNA hybrid molecule. The target can be a single-stranded or double-stranded nucleic acid. Although the most widely used PCR procedure uses a double-stranded target, this is not a necessity. After the first amplification cycle of a single-stranded DNA target, the reaction mixture contains a double-stranded DNA molecule consisting of the single-stranded target and a newly synthesized complementary strand. Similarly, following the first amplification cycle of an RNA/cDNA target, the reaction mixture contains a double-stranded cDNA molecule. At this point, successive cycles of amplification proceed as described above.

Suitable nucleic acid detection methods are known to the expert in the field and are described in standard textbooks as Sambrook J. et al., Molecular Cloning: A Laboratory Manual, Cold Spring Harbor Laboratory Press, Cold Spring Harbor, N.Y., 1989 and Ausubel F. et al.: Current Protocols in Molecular Biology 1987, J. Wiley and Sons, NY. There may be also further purification steps before the nucleic acid detection step is carried out as e.g. a precipitation step. The detection methods may include but are not limited to the binding or intercalating of specific dyes as ethidium bromide which intercalates into the double-stranded DNA and changes its fluorescence thereafter. The purified nucleic acid may also be separated by electrophoretic methods optionally after a restriction digest and visualized thereafter. There are also probe-based assays which exploit the oligonucleotide hybridization to specific sequences and subsequent detection of the hybrid.

The amplified target nucleic acids can be detected during or after the amplification reaction in order to evaluate the result of the analysis. Particularly for detection in real time, it is advantageous to use nucleic acid probes. Thus, an aspect of the invention is the process described above, wherein a cycling step comprises an amplification step and a hybridization step, said hybridization step comprising hybridizing the amplified nucleic acids with probes.

It can be favorable to monitor the amplification reaction in real time, i.e. to detect the target nucleic acids and/or their amplificates during the amplification itself. Therefore, an aspect of the invention is the process described above, wherein the probes are labeled with a donor fluorescent moiety and a corresponding acceptor fluorescent moiety.

The methods set out above can be based on Fluorescence Resonance Energy Transfer (FRET) between a donor fluorescent moiety and an acceptor fluorescent moiety. A representative donor fluorescent moiety is fluorescein, and representative corresponding acceptor fluorescent moieties include LC-Red 640, LC-Red 705, Cy5, Cy5.5, and Quasar705. Typically, detection includes exciting the sample at a wavelength absorbed by the donor fluorescent moiety and visualizing and/or measuring the wavelength emitted by the corresponding acceptor fluorescent moiety. In the process according to the invention, detection can be followed by quantitating the FRET. For example, detection is performed after each cycling step. For example, detection is performed in real time. By using commercially available real-time PCR instrumentation (e.g., LightCycler™ or TaqMan®), PCR amplification and detection of the amplification product can be combined in a single closed cuvette with dramatically reduced cycling time. Since detection occurs concurrently with amplification, the real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory.

The following patent applications describe real-time PCR as used in the LightCycler™ technology: WO 97/46707, WO 97/46714 and WO 97/46712. The LightCycler™ instrument is a rapid thermal cycler combined with a microvolume fluorometer utilizing high quality optics. This rapid thermocycling technique uses thin glass cuvettes as reaction vessels. Heating and cooling of the reaction chamber are controlled by alternating heated and ambient air. Due to the low mass of air and the high ratio of surface area to volume of the cuvettes, very rapid temperature exchange rates can be achieved within the thermal chamber.

TaqMan® technology utilizes a single-stranded hybridization probe labeled with two fluorescent moieties. When a first fluorescent moiety is excited with light of a suitable wavelength, the absorbed energy is transferred to a second fluorescent moiety according to the principles of FRET. The second fluorescent moiety is generally a quencher molecule. Typical fluorescent dyes used in this format are for example, among others, FAM, HEX, CY5, JA270, Cyan, CY5.5, and Quasar705. During the annealing step of the PCR reaction, the labeled hybridization probe binds to the target nucleic acid (i.e., the amplification product) and is degraded by the 5′ to 3′ exonuclease activity of the Taq or another suitable polymerase as known by the skilled artisan, such as a mutant Z05 polymerase, during the subsequent elongation phase. As a result, the excited fluorescent moiety and the quencher moiety become spatially separated from one another. As a consequence, upon excitation of the first fluorescent moiety in the absence of the quencher, the fluorescence emission from the first fluorescent moiety can be detected.

In both detection formats described above, the intensity of the emitted signal can be correlated with the number of original target nucleic acid molecules.

As an alternative to FRET, an amplification product can be detected using a double-stranded DNA binding dye such as a fluorescent DNA binding dye (e.g., SYBRGREEN I® or SYBRGOLD® (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

Molecular beacons in conjunction with FRET can also be used to detect the presence of an amplification product using the real-time PCR methods of the invention. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g. a hairpin). As a result of secondary structure formation within the probe, both fluorescent moieties are in spatial proximity when the probe is in solution. After hybridization to the amplification products, the secondary structure of the probe is disrupted and the fluorescent moieties become separated from one another such that after excitation with light of a suitable wavelength, the emission of the first fluorescent moiety can be detected.

Thus, in a method according to the invention is the method described above using FRET, wherein said probes comprise a nucleic acid sequence that permits secondary structure formation, wherein said secondary structure formation results in spatial proximity between said first and second fluorescent moiety.

Efficient FRET can only take place when the fluorescent moieties are in direct local proximity and when the emission spectrum of the donor fluorescent moiety overlaps with the absorption spectrum of the acceptor fluorescent moiety.

Thus, in an embodiment, said donor and acceptor fluorescent moieties are within no more than 5 nucleotides of each other on said probe. In a further embodiment, said acceptor fluorescent moiety is a quencher.

As described above, in the TaqMan format, during the annealing step of the PCR reaction, the labeled hybridization probe binds to the target nucleic acid (i.e., the amplification product) and is degraded by the 5′- to 3′-exonuclease activity of the Taq or another suitable polymerase as known by the skilled artisan, such as a mutant Z05 polymerase, during the subsequent elongation phase.

Thus, in an embodiment, in the process described above, amplification employs a polymerase enzyme having 5′- to 3′-exonuclease activity.

It is further advantageous to carefully select the length of the amplicon that is yielded as a result of the process described above. Generally, relatively short amplicons increase the efficiency of the amplification reaction. Thus, an aspect of the invention is the process described above, wherein the amplified fragments comprise up to 450 bases, up to 300 bases, up to 200 bases, or up to 150 bases.

An internal control nucleic acid can be competitive, non-competitive or partially competitive. A competitive internal control nucleic acid carries essentially the same primer binding sites as the target and thus competes for the same primers with the target. While this principle allows a good mimicry of the respective target nucleic acid due to their similar structure, it can lower the amplification efficiency with regard to the target nucleic acid or acids and thus lead to a less sensitive assay.

A non-competitive internal control nucleic acid has different primer binding sites than the target and thus binds to different primers. Advantages of such a setup comprise, among others, the fact that the single amplification events of the different nucleic acids in the reaction mixture can take place independently from each other without any competition effects. Thus, no adverse effects occur regarding the limit of detection of the assay as can be the case in a competitive setup. Finally, in an amplification reaction using a partially competitive setup the respective control nucleic acid and at least one of the target nucleic acids compete for the same primers, while at least one other target nucleic acid binds to different primers.

The fact that the method described above involves a distinct set of primers for each of said target nucleic acids and for said internal control nucleic acid renders the method considerably flexible. In this non-competitive setup it is not necessary to introduce target-specific binding sites into the control nucleic acid as in the case of a competitive setup, and the drawbacks of a competitive setup as mentioned above are avoided. In a non-competitive setup, the internal control nucleic acid has a sequence different from any target sequences, in order not to compete for their primers and/or probes. For example, the sequence of the internal control nucleic acid can be different from the other nucleic acid sequences in the fluid sample. As an example, if the fluid sample is derived from a human, the internal control nucleic acid may not have a sequence which also endogenously occurs within humans. The difference in sequence should thus be at least significant enough to not allow the binding of primers and/or probes to the respective endogenous nucleic acid or acids under stringent conditions and thus render the setup competitive. In order to avoid such interference, the sequence of the internal control nucleic acid used in the invention can be derived from a source different from the origin of the fluid sample.

Qualitative detection of a nucleic acid in a biological sample is crucial e.g. for recognizing an infection of an individual. Thereby, one important requirement for an assay for detection of a microbial infection is that false-negative or false-positive results be avoided, since such results would almost inevitably lead to severe consequences with regard to treatment of the respective patient. Thus, especially in PCR-based methods, a qualitative internal control nucleic acid is added to the detection mix. Said control is particularly important for confirming the validity of a test result: At least in the case of a negative result with regard to the respective target nucleic acid, the qualitative internal control reaction has to perform reactive within given settings, i.e. the qualitative internal control must be detected, otherwise the test itself is considered to be inoperative. However, in a qualitative setup, said qualitative internal control does not necessarily have to be detected in case of a positive result. For qualitative tests, it is especially important that the sensitivity of the reaction is guaranteed and therefore strictly controlled As a consequence, the concentration of the qualitative internal control must be relatively low so that even in a situation e.g. of slight inhibition the qualitative internal control is not be detected and therefore the test is invalidated.

Thus, an aspect of the invention is the process described above, wherein the presence of an amplification product of said internal control nucleic acid is indicative of an amplification occurring in the reaction mixture even in the absence of amplification products for one or more of said target nucleic acids.

On the other hand and in addition to mere detection of the presence or absence of a nucleic acid in a sample, it is often important to determine the quantity of said nucleic acid. As an example, stage and severity of a viral disease may be assessed on the basis of the viral load. Further, monitoring of any therapy requires information on the quantity of a pathogen present in an individual in order to evaluate the therapy's success. For a quantitative assay, it is necessary to introduce a quantitative standard nucleic acid serving as a reference for determining the absolute quantity of a target nucleic acid. Quantitation can be effectuated either by referencing to an external calibration or by implementing an internal quantitative standard.

In the case of an external calibration, standard curves are created in separate reactions using known amounts of identical or comparable nucleic acids. The absolute quantity of a target nucleic acid is subsequently determined by comparison of the result obtained with the analyzed sample with said standard function. External calibration, however, has the disadvantage that a possible extraction procedure, its varied efficacy, and the possible and often not predictable presence of agents inhibiting the amplification and/or detection reaction are not reflected in the control.

This circumstance applies to any sample-related effects. Therefore, it might be the case that a sample is judged as negative due to an unsuccessful extraction procedure or other sample-based factors, whereas the target nucleic acid to be detected and quantified is actually present in the sample.

For these and other reasons, an internal control nucleic acid added to the test reaction itself is of advantage. When serving as a quantitative standard, said internal control nucleic acid has at least the following two functions in a quantitative test:

i) It monitors the validity of the reaction.
ii) It serves as reference in titer calculation thus compensating for effects of inhibition and controlling the preparation and amplification processes to allow a more accurate quantitation. Therefore, in contrast to the qualitative internal control nucleic acid in a qualitative test which must be positive only in a target-negative reaction, the quantitative control nucleic acid in a quantitative test has two functions: reaction control and reaction calibration. Therefore it must be positive and valid both in target-negative and target-positive reactions.

It further has to be suited to provide a reliable reference value for the calculation of high nucleic acid concentrations. Thus, the concentration of an internal quantitative control nucleic acid needs to be relatively high.

The internal control nucleic acid used in the present invention can serve as a “quantitative standard nucleic acid” which is apt to be and used as a reference in order to quantify, i.e. to determine the quantity of the target nucleic acids. For this purpose, one or more quantitative standard nucleic acids undergo all possible sample preparation steps along with the target nucleic acids. Moreover, a quantitative standard nucleic acid is processed throughout the method within the same reaction mixture. It must generate, directly or indirectly, a detectable signal both in the presence or absence of the target nucleic acid. For this purpose, the concentration of the quantitative standard nucleic acid has to be carefully optimized in each test in order not to interfere with sensitivity but in order to generate a detectable signal also e.g. at very high target concentrations. In terms of the limit of detection (LOD, see below) of the respective assay, the concentration range for the “quantitative standard nucleic acid” is 20-5000×LOD, 20-1000×LOD, or 20-5000×LOD. The final concentration of the quantitative standard nucleic acid in the reaction mixture is dependent on the quantitative measuring range accomplished.

“Limit of detection” or “LOD” means the lowest detectable amount or concentration of a nucleic acid in a sample. A low “LOD” corresponds to high sensitivity and vice versa. The “LOD” is usually expressed either by means of the unit “cp/ml”, particularly if the nucleic acid is a viral nucleic acid, or as IU/ml. “Cp/ml” means “copies per milliliter” wherein a “copy” is copy of the respective nucleic acid. IU/ml stands for “International units/ml”, referring to the WHO standard.

A widely used method for calculating an LOD is “Probit Analysis”, which is a method of analyzing the relationship between a stimulus (dose) and the quantal (all or nothing) response. In a typical quantal response experiment, groups of animals are given different doses of a drug. The percent dying at each dose level is recorded. These data may then be analyzed using Probit Analysis. The Probit Model assumes that the percent response is related to the log dose as the cumulative normal distribution. That is, the log doses may be used as variables to read the percent dying from the cumulative normal. Using the normal distribution, rather than other probability distributions, influences the predicted response rate at the high and low ends of possible doses, but has little influence near the middle.

The Probit Analysis can be applied at distinct “hitrates”. As known in the art, a “hitrate” is commonly expressed in percent [%] and indicates the percentage of positive results at a specific concentration of an analyte. Thus for example, an LOD can be determined at 95% hitrate, which means that the LOD is calculated for a setting in which 95% of the valid results are positive.

Further, in the sense of the invention, the internal control nucleic acid can serve as a “qualitative internal control nucleic acid”. A “qualitative internal control nucleic acid” is particularly useful for confirming the validity of the test result of a qualitative detection assay: Even in the case of a negative result, the qualitative internal control must be detected, otherwise the test itself is considered to be inoperative. However, in a qualitative setup, it does not necessarily have to be detected in case of a positive result. As a consequence, its concentration must be relatively low. It has to be carefully adapted to the respective assay and its sensitivity. For example, the concentration range for the qualitative internal nucleic acid, i.e. the second control nucleic acid, will comprise a range of 1 copy per reaction to 1000 copies per reaction. In relation to the respective assay's limit of detection (LOD), its concentration is between the LOD of an assay and the 25 fold value of the LOD, or between the LOD and 10×LOD. Or, it is between 2× and 10×LOD. Or, it is between 5× and 10×LOD. Or, it is 5× or 10×LOD.

The internal control nucleic acid as used in the present invention is not restricted to a particular sequence. It can be advantageous to add different internal control nucleic acids to a fluid samples, but to use only one of them for amplification e.g. by adding only primers for one of said internal control nucleic acids. In such embodiments, the internal control nucleic acid to be amplified in a certain experiment can be chosen by the person skilled in the art, thus increasing flexibility of the analysis to be carried out. In particularly advantageous embodiments, said different internal control nucleic acids can be comprised by a single nucleic acid construct, e.g. a plasmid or a different suitable nucleic acid molecule.

The following examples are given to illustrate embodiments of the present invention as it is presently preferred to practice. It will be understood that the examples are illustrative, and that the invention is not be considered as restricted except as indicated in the appended claims.

### EXAMPLES

### Example 1: Quasar 705 Fluorescent Dye

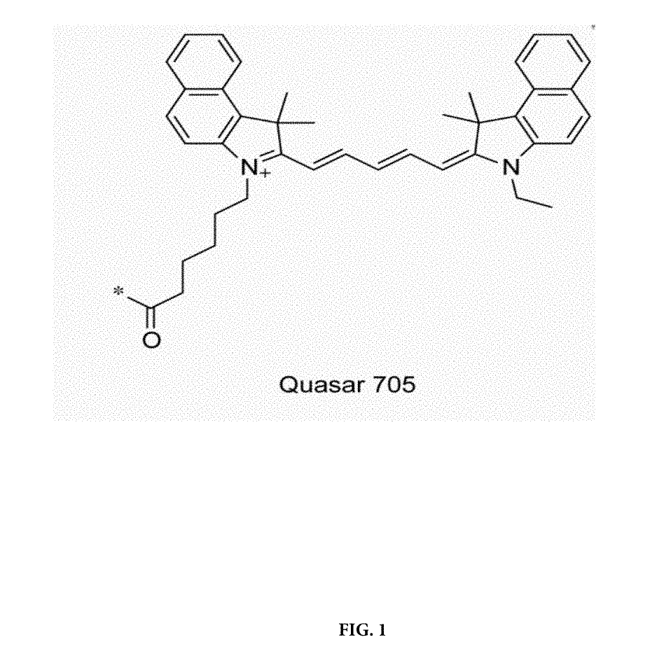

Quasar 705 (LGC Biosearch Technologies, Petaluma, Calif.) is an indocarbocyanine dye that fluoresces in the red region of the visible spectrum with an absorption maximum at 690 nm and emission maximum at 705 nm. FIG. 1 shows the chemical structure of Quasar 705. The synthesis and coupling of Quasar 705 to nucleoside moieties are described in U.S. Pat. No. 8,436,153 which is incorporated herein by reference in its entirety.

### Example 2: Real-Time PCR Assay Conditions

Primers, probes, and kits designed for the detection of Trichomonas vaginalis (TV) and of Mycoplasma genitalium (MG) are described in U.S. Provisional Patent Application No. 62/342,600, titled “Compositions and methods for detection of Trichomonas vaginalis” and U.S. Provisional Patent Application No. 62/342,519, titled “Compositions and methods for detection of Mycoplasma genitalium”, respectively, which are both incorporated herein by reference in their entireties. Real-time PCR detection of TV and MG were performed using either the Cobas® 4800 system or the LightCycler® 480 system (Roche Molecular Systems, Inc., Pleasanton, Calif.). The final concentrations of the amplification reagents are shown below:

TABLE I
PCR Amplification Reagents

| Master Mix Component | Final Conc (50 uL) | |
|---|---|---|
| NaN3 | 0.030% | |
| Potassium acetate | 120.0 | mM |
| Glycerol | 3.0% | |
| Tricine | 60.0 | mM |
| Aptamer | 0.18 | uM |
| UNG Enzyme | 5.0 | U |
| Z05-SP-PZ Polymerase | 30.0 | U |
| dATP | 521.70 | uM |
| dCTP | 521.70 | uM |
| dGTP | 521.70 | uM |
| dUTP | 1043.40 | uM |
| Forward primer oligonucleotides-target | 0.10-0.30 | μM |
| Reverse primer oligonucleotides-target | 0.15-0.30 | μM |
| Probe oligonucleotides-target | 0.10 | μM |
| Forward primer oligonucleotides-IC | 0.50 | μM |
| Reverse primer oligonucleotides-IC | 0.50 | μM |
| Probe oligonucleotides-IC | 0.10 | μM |
| Manganese Acetate | 3.80 | mM |

The following table shows the typical thermoprofile used for PCR amplification reaction

TABLE II
PCR Thermoprofile

| Step | Target (° C.) | Hold time (mm:ss) | Ramp Rate (° C./s) Cycles | |
|---|---|---|---|---|
| UNG | 50 | 02:00 | 4.4 | 1 |
| Pre-Cycle | 95 | 05:00 | 4.4 | 1 |
| Denaturation | 95 | 00:10 | 4.4 | 50 |
| Annealing | 59 | 00:35 | 2.2 | 50 |
| Cooling | 40 | 00:30 | 2.2 | 1 |

### Example 3: Results TV PCR Assay

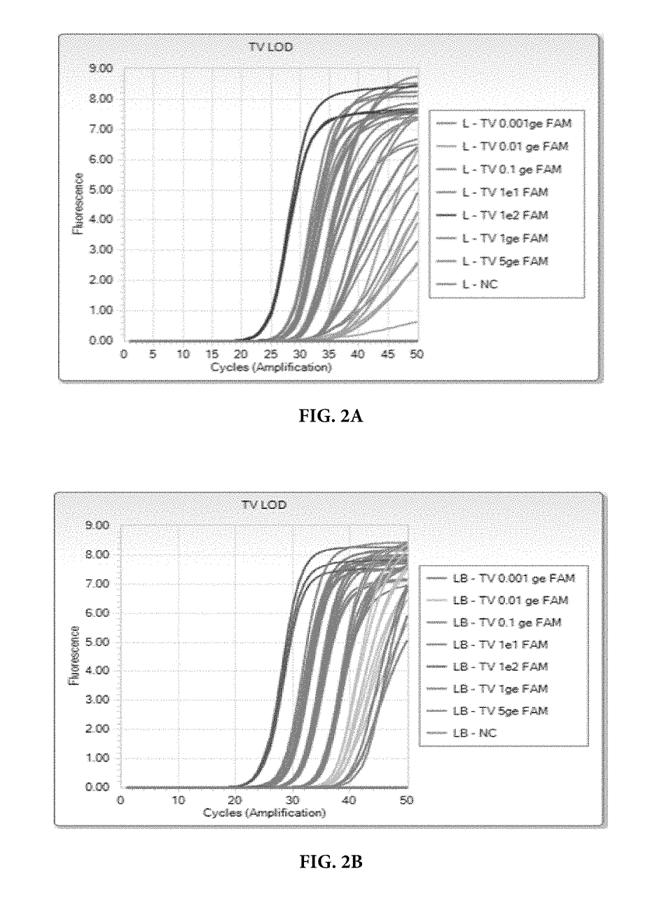

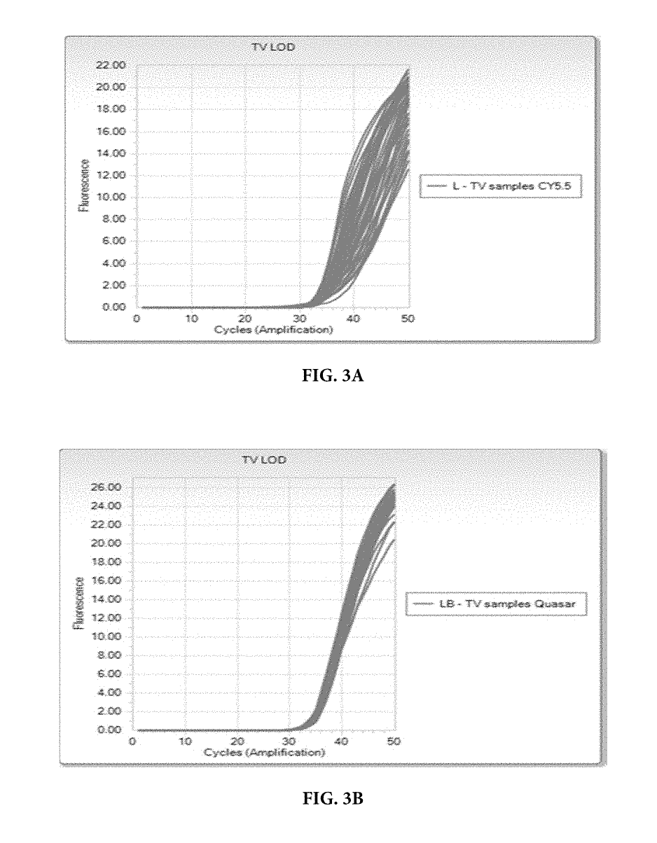

Two master mixes were compared in a side by side experiment for TV's Limit of detection (LOD), one in which a Cy 5.5 Internal Control (IC) probe was used and one in which a Quasar 705 IC probe was used. The TV-specific probe was labeled with FAM. The TV LOD test included TV genomic DNA in 8-replicates at 0.001, 0.01, 0.1, 1, 5 genomes per PCR levels and 3 replicates for 10 and 100 genomes per PCR level in the presence of IC plasmid at 35 copies per PCR reaction. The results showed 100 percent hits for TV target up to the 0.01 ge level and dropouts were seen only at 0.001 ge level (FIG. 2A and FIG. 2B). Hence the TV LOD with both master mixes is 0.01 ge per PCR. The dropout rate at 0.001 ge level was 8 out of 8 for the master mix with Cy5.5 IC probe and 4 out of 8 for the master mix with Quasar 705 IC probe. The growth curves with the Quasar 705 IC probe are more robust and the threshold cycle (Ct) values are tighter (compare FIG. 2A and FIG. 2B). The PCR assay with the Quasar 705 IC probe was able to detect several replicates at 0.001 ge per PCR level whereas the assay using Cy5.5 IC probe did of detect any replicates at 0.001 ge per PCR level. The growth curves for the internal control nucleic acid in channel 4 (IC) are shown on FIG. 3A and FIG. 3B. The results show that the growth curves are more robust with tighter Ct values using the Quasar 705 IC probe (FIG. 3B) compared to the Cy5.5 IC probe (FIG. 3A).

### Example 4: Results MG PCR Assay

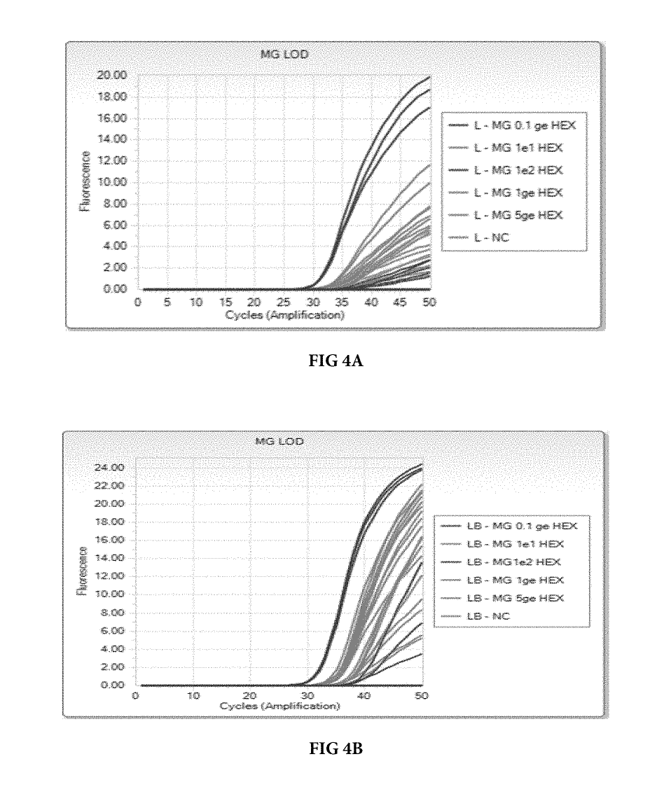

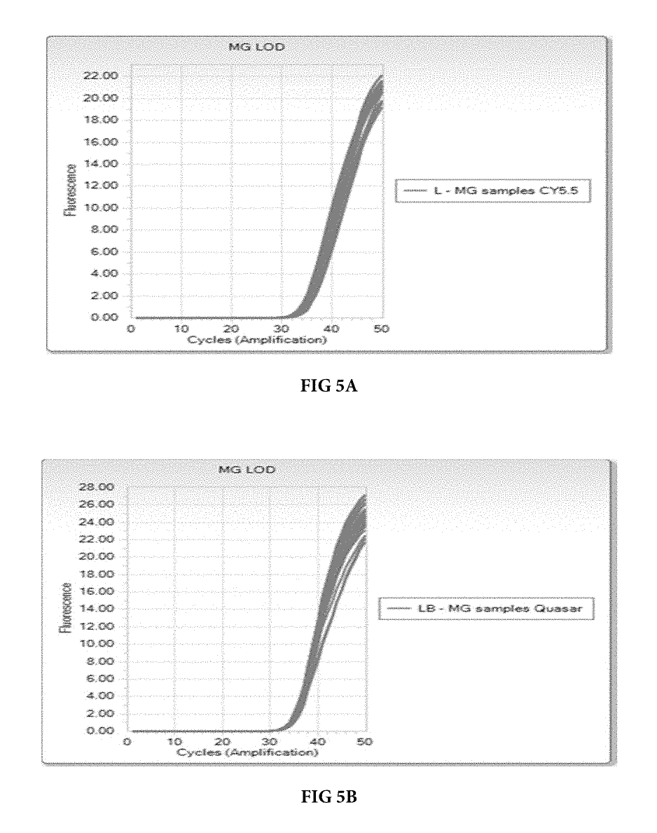

Two master mixes were compared in a side by side experiment for MG's Limit of detection (LOD), one in which a CY5.5 IC probe was used and one in which a Quasar 705 IC probe was used. The MG-specific probe was labeled with HEX. The MG LOD test included MG genomic DNA in 8-replicates at 0.01, 0.1, 1, 5 ge per PCR levels and 3 replicates for 10 and 100 ge per PCR level in presence of 35 copies of IC plasmid per PCR. The results showed 100 percent hits up to the 1 ge level and dropouts were seen at 0.1 ge level (FIG. 4A and FIG. 4B). Hence the MG LOD with both master mixes is 1 ge per PCR. The dropout rate at 0.1 ge level was 3 out of 8 for the master mix with Cy5.5 IC probe and 5 out of 8 for the master mix with Quasar 705 IC probe making 1 ge/PCR the limit of detection for MG in a clean target system. Similar to the comparison test for TV LOD, the RFI's with master mixes using the Quasar 705 IC probe (FIG. 5B) are also higher and the Ct values are tighter than master mixes using the Cy5.5 IC probe (FIG. 5A) in the IC channel.

What is claimed is:

1. A method for detecting two or more target nucleic acids in a sample comprising the steps of:
a) providing a reaction mixture comprising
an internal control (IC) nucleic acid;
two or more target-specific primer pairs that hybridize to distinct sequence portions of the two or more target nucleic acid;
an IC-specific primer pair that hybridize to distinct sequence portions of the IC nucleic acid;
two or more target-specific probes wherein each one target-specific probe is labeled with a fluorescent dye that is different from another target-specific probe, and wherein each one of the two or more target-specific probes specifically hybridize to each one of the two or more target nucleic acid sequences amplified by each one of the two or more target-specific primer pairs;
an IC-specific probe labeled with Quasar 705 that hybridizes to the IC nucleic acid sequence amplified by the IC-specific primer pair;
b) adding the sample to the reaction mixture;
c) performing one or more cycling steps, wherein each cycling step comprises:
an amplifying step comprising producing two or more amplification products derived from the two or more target nucleic acids if present in the sample and producing an amplification product derived from the IC nucleic acid; and
a hybridizing step comprising hybridizing amplification products with probes to generate fluorescent signals;
d) detecting and measuring signals generated from each fluorescent dye on the two or more target-specific probes and from Quasar 705 on the IC-specific probe in step c), wherein the presence or absence of fluorescent signals generated from the target-specific probes are indicative of the presence or absence of the target nucleic acids;
wherein the sensitivity and intensity of the signals generated from the fluorescent dyes on the target-specific probes is improved when the IC-specific probe is labeled with Quasar 705 than when the IC-specific probe is labeled with Cy5.5.

2. The method of claim 1, wherein the fluorescent dye on each of the target-specific probes is a fluorescein dye, a rhodamine dye, a cyanine dye, and a coumarin dye.

3. The method of claim 2, wherein the fluorescent dye on each of the target-specific probes is selected from Fluorescein (FAM) and/or), Hexachloro-fluorescein (HEX).), JA270, CAL635, Coumarin343, Cyan500, CY5.5, LC-Red 640, and/or LC-Red 705.

4. The method of claim 3, wherein the fluorescent dye on each of the target-specific probes is selected from Fluorescein (FAM) and/or Hexachloro-fluorescein (HEX).

5. The method of claim 1, wherein any one or more of the primers and/or probes comprises a modified nucleotide or a non-nucleotide compound.

6. The method of claim 1, wherein at least one of the two or more target nucleic acid sequences is from one or more DNA virus or from one or more bacterium.

7. The method of claim 6, wherein at least one of the two or more target nucleic acid sequences is from Chlamydia trachomatis (CT), Neisseria gonorrhoeae (NG), Trichomonas vaginalis (TV) and/or Mycoplasma genitalium (MG).

8. The method of claim 7 wherein the two or more target nucleic acid sequences are from Trichomonas vaginalis (TV) and Mycoplasma genitalium (MG).

9. The method of claim 1, wherein said internal control nucleic acid is DNA.

10. The method of claim 1, wherein at least one of the two or more target nucleic acid sequences is from one or more RNA virus.

11. The method of claim 10, wherein the one or more RNA virus is selected from any one of Human Immunodeficiency Virus (HIV), Hepatitis C Virus (HCV), the West Nile Virus (WNV), Human Papilloma Virus (HPV), Japanese Encephalitis Virus (JEV), and/or St. Louis Encephalitis Virus (SLEV).

12. The method of any one of claim 1, wherein said internal control nucleic acid is RNA.
